(19) United States
(12) Patent Application Publication
(10) Pub. No.: US 2025/0199148 A1
(43) Pub. Date: Jun. 19, 2025

(54) METHOD AND SYSTEM FOR TRANSCRANIAL ULTRASOUND IMAGING (TUI)

(71) Applicant: TECHNISCHE UNIVERSITEIT DELFT, Delft (NL)

(72) Inventors: Moein MOZAFFARZADEH, Delft (NL); Nicolaas DE JONG, Delft (NL); Guillaume Gérard Joseph RENAUD, Delft (NL); Martin Daniël VERWEIJ, Delft (NL)

(73) Assignee: TECHNISCHE UNIVERSITEIT DELFT, Delft (NL)

(21) Appl. No.: 18/842,963

(22) PCT Filed: Mar. 3, 2023
(86) PCT No.: PCT/EP2023/055399

§ 371 Date: Aug. 30, 2024

(30) Foreign Application Priority Data
Mar. 3, 2022 (NL) 2031149

(51) Int. Cl. G01S 7/52; A61B 8/00; A61B 8/08

(52) U.S. Cl. CPC G01S 7/52049; A61B 8/0808; A61B 8/461; G01S 7/52053

(57) ABSTRACT

Provided herein is a method for transcranial ultrasound imaging (TUI), using sending an ultrasound wave to a target imaging area through a superficial area and a first element, detecting ultrasound waves reflected or backscattered from the target imaging area. Estimates are used of the wave speed in the superficial area, in the first element and in the target imaging area and the position and geometry of the near surface and far surface of the first element are determined. A phase aberration correction is applied which includes the evaluation of the travel times for multiple ultrasound ray paths passing through intermediate points defined on the near and far surfaces of the first element, and determining the correct ultrasound ray path by selecting the ultrasound ray path with the shortest travel time.

### FIELD OF THE INVENTION

The present invention relates to a method for transcranial ultrasound imaging (TUI), comprising sending an ultrasound signal to a target imaging area (e.g. a brain) through a first element (e.g. a skull bone part forming an aberration layer) having a medium with a wave-speed different from the wave-speed in the medium in the target imaging area; and detecting ultrasound waves reflected or backscattered from the target imaging area through the first element.

### BACKGROUND ART

European patent publication EP-A-3594678 discloses a method and system for examining the interior material of an object, such as a pipeline or a human body, from a surface of the object using ultrasound.

International patent publication WO2014/200417 discloses a method and system for determining a property of a non-homogeneous material using sound waves, allowing to determine a local property in a non-homogenous material from a surface of the material.

US patent publication US2018/0116632 discloses system and method embodiments of portable ultrasonic imaging devices, more in particular acoustic power scalable charge-redistribution ultrasonic transducer interfaces with on-chip aberration compensation.

US patent publication US2020/0359992 discloses a method for characterizing bone by receiving ultrasonic wave echo signals transmitted into a body. A speed of sound in the body's non-bone biological tissue is determined, and a first demarcation curve between non-bone biological tissue and bone is located in an image of the body constructed during said determining step, and a speed of sound in bone is determined. The steps of determining speed include constructing images from the signals, and a metric calculation indicative of a focus quality in the constructed images.

### SUMMARY OF THE INVENTION

The present invention seeks to provide an improved ultrasound imaging method and system, which is particularly suitable for imaging a target area through a first element having a wave-speed different from the wave-speed in the medium in the target imaging area, such as a temporal bone of a human skull in transcranial ultrasound imaging. The first element can also be seen as an aberrating layer having an (an) isotropic velocity model.

According to the present invention, a method as defined above is provided, further comprising sending an ultrasound wave to a target imaging area through a superficial area and a first element, where the target imaging area, the superficial area and the first element have different ultrasound wave speeds;
detecting ultrasound waves reflected or backscattered from the target imaging area through the first element and the superficial area, and detecting ultrasound waves reflected or backscattered from the first element through the superficial area; and
processing the detected ultrasound waves by:
   using estimates of the wave speed in the superficial area, in the first element and in the target imaging area; and
   using the detected ultrasound waves reflected or backscattered from the first element through the superficial area for forming the ultrasound image of the superficial area up to a near surface of the first element; and
   determining the position and geometry of the near surface of the first element; and
   applying a phase aberration correction to the detected ultrasound waves reflected or backscattered from the first element by defining intermediate points on the near surface of the first element to test different ultrasound ray paths, for forming the ultrasound image of the first element up to a far surface of the first element, using the estimates of wave speed, and the position and geometry of the near surface of the first element; and
   determining the position and geometry of the far surface of the first element; and
   applying a phase aberration correction to the detected ultrasound waves reflected or backscattered from the target imaging area by defining intermediate points on the far surface of the first element to test different ultrasound ray paths, for forming the ultrasound image of the target imaging area using the estimates of wave speed, and the position and geometry of the near and far surfaces of the first element.
wherein the phase aberration correction includes the evaluation of the travel times for multiple ultrasound ray paths passing through the intermediate points defined on the near and far surfaces of the first element, and determines the correct ultrasound ray path by selecting the ultrasound ray path with the shortest travel time.

### SHORT DESCRIPTION OF DRAWINGS

The present invention will be discussed in more detail below, with reference to the attached drawings, in which

FIG. 1A-C show schematic views of various stages in ray-tracing steps applied in a method embodiment of the present invention;

FIG. 2 shows a block diagram of a system according to an exemplary embodiment of the present invention; and

FIG. 3A-C show images obtained using one of the present invention embodiments.

### DESCRIPTION OF EMBODIMENTS

Transcranial ultrasound imaging (TUI) is a safe and relatively inexpensive diagnostic modality with numerous applications such as stroke prevention and diagnosis and detection of vasospasm after subarachnoid hemorrhage (most often caused by head trauma). While TUI is available in hospitals and emergency medicine services (EMS) nowadays, it is still hindered by the low image quality caused by the strong wave aberration and multiple scattering caused by the skull.

TUI is often performed through the temporal window (the thinnest part of the skull that gives the most optimal ultrasound access to the brain) where the squamous part of the temporal bone often consists of a single layer of cortical bone. To deal with the phase aberration, the temporal bone can be modeled as an infinitesimally thin aberrating layer at the surface of the transducer (so called the near-field phase-screen aberration model), but the correction obtained by this approach is only limited to certain regions called the isoplanatic patches. Another approach is using either ultrasound measurements or CT/MRI scans of the skull to obtain the true geometry and sound speed of the temporal bone prior to image reconstruction, and then correct refraction during image reconstruction.

Single-sided two-dimensional transcranial ultrasound through the human temporal window using a single handheld commercial probe, seems possible if the position, true geometry and sound speed of the bone layer are estimated for an accurate phase aberration correction. However, no fast-enough implementation is available for real-time transcranial imaging, which limits its application in practice, especially for translation to 3D TUI. This is mainly because the normally used algorithms for such a real-time imaging application are iterative and a fast-enough implementation is not available.

In the present invention embodiments, a method is provided for transcranial ultrasound imaging (TUI, comprising:
sending an ultrasound wave to a target imaging area 6 through a superficial area 4 and a first element 5, where the target imaging area 6, the superficial area 4 and the first element 5 have different ultrasound wave speeds;
detecting ultrasound waves reflected or backscattered from the target imaging area 6 through the first element 5 and the superficial area 4, and detecting ultrasound waves reflected or backscattered from the first element 5 through the superficial area 4; and processing the detected ultrasound waves by:
   using estimates of the wave speed in the superficial area 4, in the first element 5 and in the target imaging area 6; and
   using the detected ultrasound waves reflected or backscattered from the first element 5 through the superficial area 4 for forming the ultrasound image of the superficial area 4 up to a near surface of the first element 5; and
   determining the position and geometry of the near surface of the first element 5; and
   applying a phase aberration correction to the detected ultrasound waves reflected or backscattered from the first element 5 by defining intermediate points on the near surface of the first element 5 to test different ultrasound ray paths, for forming the ultrasound image of the first element 5 up to a far surface of the first element 5, using the estimates of wave speed, and the position and geometry of the near surface of the first element 5; and
   determining the position and geometry of the far surface of the first element 5; and
   applying a phase aberration correction to the detected ultrasound waves reflected or backscattered from the target imaging area 6 by defining intermediate points on the far surface of the first element 5 to test different ultrasound ray paths, for forming the ultrasound image of the target imaging area 6 using the estimates of wave speed, and the position and geometry of the near and far surfaces of the first element 5,
wherein the phase aberration correction includes the evaluation of the travel times for multiple ultrasound ray paths passing through the intermediate points defined on the near and far surfaces of the first element 5, and determines the correct ultrasound ray path by selecting the ultrasound ray path with the shortest travel time.

The first element 5 is e.g. a bone layer, which is irregularly shaped and has a wave-speed for the ultrasound waves travelling through it different from the wave-speed in the medium in the target imaging area. The present invention method embodiments allow a correction of wave aberration caused by the first element 5 (such as an aberrating layer 5 in a bone), e.g. acoustic wave refraction caused by the human temporal bone. By applying a resource-efficient ray-tracing technique, the method can be applied in real-time for transcranial ultrasound imaging. It is noted that the present invention embodiments can be applied in further ultrasound imaging techniques, such as but not limited to transcranial photoacoustic imaging, either in microscopic or mesoscopic level.

In the present invention embodiments, the first element 5 (or the aberrating layer therein) can be a flat, curved or irregularly-shaped object. The first element 5 (or aberrating layer therein) can also have a three-layer structure (e.g. a layering of cortical bone-trabecular bone-cortical bone). The element 4 can also have a wave-speed different from the wave-speed in the first element (5) and the medium in the target imaging area (6).

In a further embodiment, a method is provided wherein the ultrasound waves are sent and detected using an ultrasound probe 2 having a first plurality Nt of transmitter elements and a second plurality Nr of receiver elements, and the phase aberration correction/ray-tracing technique comprises a two-point ray tracing technique determining a shortest travel time connecting a pixel in the element 4, or inside the first element 5, or in the target imaging area 6, to a source or receiver element of the ultrasound probe 2, for each of the plurality Nt of sources and for each of the plurality Nr of receiver elements, via one intermediate point per crossed interface. intermediate points are pixels of the image. It is noted that the first plurality Nt of transmitter elements can be equal to the second plurality Nr of receiver elements, but can also be different numbers. The basics of such a resource-efficient ray-tracing method is known as such, e.g. referred to as Waltham concept, see D. Waltham, “Two-point ray tracing using Fermat's principle,” Geophysical Journal International, vol. 93, no. 3, pp. 575-582, 1988.

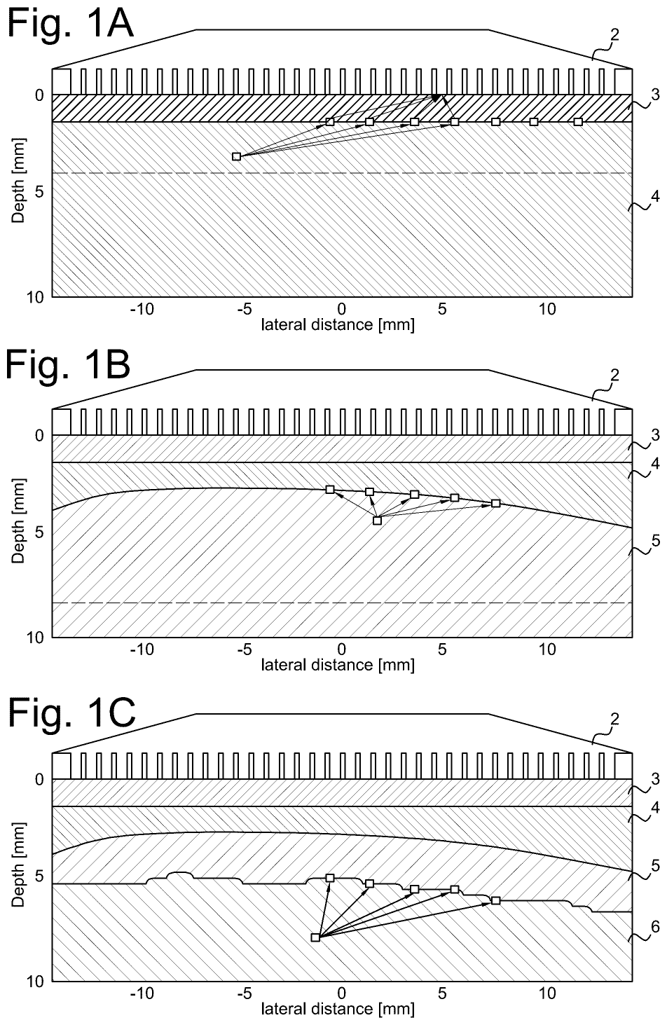

An exemplary embodiment of this method is discussed below with reference to FIG. 1A-C, which each show one of the three steps of the ray-tracing method. The FIGS. 1A-C show a cross sectional view with depth and lateral distance as vertical and horizontal axis, respectively. A compressional (acoustic) wave travels from an image pixel (in the depth-lateral distance dimensions as shown) to a receiver element of the ultrasound probe (or transducer) 2 following a path that corresponds to the shortest travel-time. So in other words, the ultrasound waves are sent and detected using an ultrasound probe 2 having a first plurality Nt of sources and a second plurality Nr of receiver elements, and the ray-tracing technique comprises a two-point ray tracing technique determining a shortest travel time connecting a pixel in the element (4), or inside the first element (5), or in the target imaging area (6), to a source or receiver element of the ultrasound probe (2), for each of the plurality Nt of sources and for each of the plurality Nr of receiver elements, via one intermediate point per crossed interface. intermediate points are pixels of the image.

Note that in the FIG. 1A-C a practical implementation is shown with an ultrasound probe 2 in combination with an acoustic lens 3, wherein a target area 6 (e.g. the brain) is imaged through an aberrating layer (first element 5, e.g. a bone layer) and a second element 4 (e.g. skin layer). In a further embodiment of the present invention, a method is provided further including applying a phase aberration correction to the detected ultrasound signal for a second element 4, the second element 4 having predetermined acoustic properties. As mentioned, in transcranial ultrasound 10 imaging applications, the second element 4 is e.g. a layer of skin.

In a further group of embodiments, the ultrasound probe 2 further includes an acoustic lens 3, e.g. adjacent to the first plurality Nt of transmitter elements and second plurality Nr of receiver elements. The acoustic lens 3 allows for a predetermined formation of the ultrasound beam.

The schematic views in FIG. 1A-C may be seen as a cross sectional view of the combination of ultrasound probe 2 and acoustic lens 3. It is noted that in further embodiments, the ultrasound probe 2 comprises a one-dimensional array (allowing 2D imaging) or a two-dimensional array (allowing 3D imaging).

Translation of the proposed reconstruction approach for 3D transcranial ultrasound imaging is possible using a matrix array transducer and determining the three-dimensional geometry of the outer and inner surfaces of the first element 5 (skull). The strategy explained in relation to FIG. 1A-C can be used for ray-tracing in three dimensions as well. For boundary segmentation in three dimensions, the Dijkstra technique can be used in each 2D plane, resulting in a one-dimensional vector. By stacking the vectors next to each other, the 3D geometry of the first element 5 (bone layer of skull) can be estimated.

In the exemplary embodiment shown in FIG. 1A-C, the following six steps are present to reconstruct a refraction-corrected image of the target area 6 (brain), through the first element 5 (more in particular, the aberrating layer of a bone) and second element 4 (skin):
1—The pixels on the far surface of the acoustic lens 3 are defined as lens intermediate points (LIPs, the squares in FIG. 1A).
2—The ray tracing starts to find the shortest travel-time from pixels in the image area relating to the skin (second element 4), up to a certain maximum depth where the near surface of the bone (first element 5) is expected to be (see the dashed line in FIG. 1A) to each receiver element through the LIPs defined in step 1; see the vectors in FIG. 1A. The (predetermined) sound speed of the skin (second element 4) is used to convert the distance to travel-time. 3—The image is reconstructed up to the maximum expected depth of the near surface of the skull 5 and using the travel-times, calculated in step 2. Then, the near surface of the skull 5 is segmented using e.g. Dijkstra's algorithm, which seeks the path that crosses the image from left to right and follows the image pixels with the highest intensity in the ultrasound image, by maximizing a merit, in this example the sum of the pixel values along the path. The IPs are updated (called near IPs, or NIPs) to the pixels on the segmented near surface (see the black squares in FIG. 1B). For the Dijkstra's algorithm, reference is made to the article by D. Hong, “Medical image segmentation based on accelerated Dijkstra algorithm,” in Advances in Intelligent Systems: Springer, 2012, pp. 341-348.
4—The ray tracing finds the shortest travel-time from pixels in the image of the bone (aberrating layer 5) up to a certain maximum depth where it is expected to detect the far surface of the bone 5 (see the dashed line in FIG. 1B) to each receiver element through the NIPs obtained in step 3. The vectors in FIG. 1B are examples of the paths. The compressional wave speed of the bone 5 is used to convert the distances to travel-times.
5—The image is reconstructed up to the maximum expected depth of the far surface of the bone 5 and using the travel-times calculated in step 4. The far surface of the bone 5 is segmented using Dijkstra's algorithm again, and the IPs are updated (now called Far IPs, or FIPs) to the pixels on the segmented far surface (see the squares in FIG. 1C).
6—Finally, the ray tracing finds the shortest travel-time from pixels in the brain (target area 6), up to the maximum imaging depth, to each receiver element through the FIPs obtained in step 5, and the rest of the image is reconstructed.

As an exemplary embodiment, a delay-and-sum beamforming technique is used for image reconstruction. Prior knowledge of the thickness of and wave speed in the acoustic lens 3 is used in step 1. Also the wave speed in the first element 5 (bone) is known, a value from the literature can be used or it can be estimated with e.g. the bidirectional head-wave technique known as such to the person skilled in the art. The depths of the first and second reconstruction steps (the dashed lines in FIGS. 1A and 1B, respectively) are determined by prior knowledge on the thickness of the skin and bone layers (first and second element 4, 5). Note that the terms “near surface” and “far surface” are defined with respect to the ultrasound probe 2.

In a further group of embodiments, the steps of processing are executed on a graphical processing unit, GPU 13. Such a graphical processing unit has proven to be very suitable for efficiently implementing the method of the present invention embodiments, even allowing to provide real-time imagery for transcranial ultrasound imaging application. In further alternative or additional embodiments, the steps of processing are executed on a cluster processing system, and/or a central processing unit (CPU). Also multiple types of processor architectures may be used simultaneously for further improvement of efficiency.

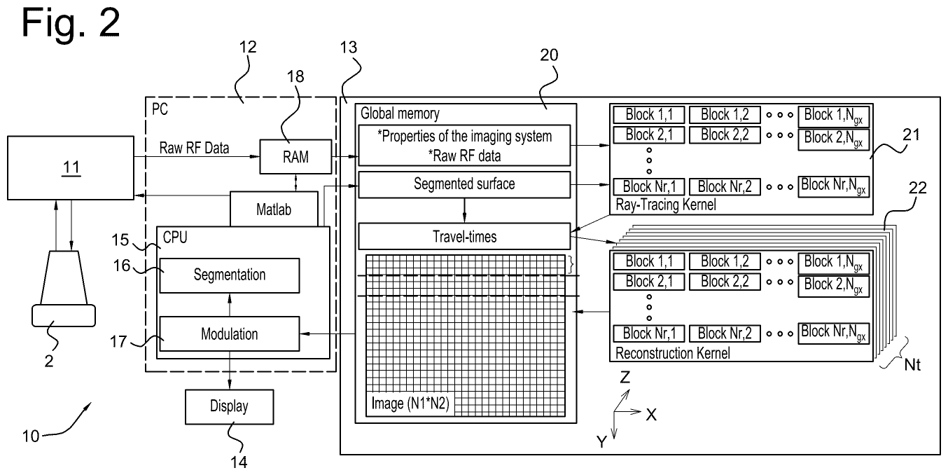

In an exemplary implementation, the approach for estimating the arrival-times and image reconstruction are implemented in a GPU 13 using the ‘compute unified device architecture’ (CUDA) platform and the application programming interface (API) model created by Nvidia. FIG. 2 shows a schematic of the hardware/software level setup. The ultrasound probe 2 (see FIG. 1A-C) is connected to an ultrasound unit 11, and provides raw RF data to a processing unit 12. The processing unit 12 interfaces with a GPU 13. In generic wording, a further aspect of the present invention relates to a system for transcranial ultrasound imaging (TUI), comprising an ultrasound unit 11 having an ultrasound probe 2 with a plurality of transmitter and receiver elements for sending and detecting an ultrasound signal, a processing unit 12 connected to the ultrasound unit 11 and arranged for data manipulation and image display, and a graphic processing unit 13 connected to the processing unit 12 and arranged to execute the method according to any one of the present invention embodiments.

The ultrasound unit 11 controls the ultrasound probe 2 for sending an ultrasound signal and detecting a reflected ultrasound signal. Once the RF data of one frame of an image to be provided is fully stored in the local memory of the ultrasound unit 11, it is transferred to a random-access memory (RAM) 18 of the processing unit 12, e.g. using a PCI express bus. The RF data is time gain compensated and filtered (based on the bandwidth of the ultrasound probe 2) during data acquisition by the ultrasound unit 11. The RF data along with the properties of the imaging system (e.g., the properties of the sound probe 2, the size of the medium, the coordinates of the pixels and primary IPs, the minimum/maximum bone thickness, etc.) are then passed to a CUDA-written Matlab executable (MEX) function being executed by the processing unit 12. The MEX function synchronously and asynchronously copies the properties of the imaging system and the pre-beamformed RF data to the GPU global memory 20, respectively, and then the image reconstruction as described above can start.

For the implementation of the imaging method, two kernels were developed, which are being executed by the GPU 13 on the data stored in the GPU global memory 20, a ray tracing kernel 21 to find the arrival-times and a reconstruction kernel 22 to form the images. These two kernels 21, 22 are utilized three times: first to find the near surface of the bone (first element 5), secondly to find the far surface of the bone (first element 5) and, finally to generate the final image. The travel-times calculated by the ray-tracing kernel 21 in the first two times are stored in the global memory 20. This strategy makes it possible to reduce the combinational search from Nip³ (Nip is the number of intermediate points (IPs) used to describe an interface) travel-times to 3Nip for each pixel in the brain/target area 6.

Thus, in a further embodiment of the present invention, the phase aberration correction/ray-tracing technique comprises applying a ray-tracing kernel 21 and a reconstruction kernel 22 consecutively, first for finding intermediate points on a near surface of the first element (bone layer), then for finding intermediate points of a far surface of the first element 5, and, finally, to generate the transcranial ultrasound image.

The MEX function mentioned above is developed based on a sampling frequency equal to four times the transducer central frequency, allowing to use a direct sampling concept (the quadrature component (Q) of the analytic signal (I/Q) is approximately the in-phase signal delayed by one data sample).

With respect to the size of the kernels 21, 22 used, relevant for the selection of capacities/capabilities of the GPU 13, the following is noted. For each transmit event (Nt in total, one for each of the first plurality of Nt transmit elements in the ultrasound probe 2), there are Nr receiver elements and N1×N2 image pixels (depth×lateral direction). One-dimensional (1D) blocks (block.x) with a size of 1024 are used in this example. The grid size used for the ray tracing kernel 21 is two dimensional and calculated based on the number of pixels desired for the eventual image, block size and number of receivers as: Ngx=NOP/block.x+((NOP % block.x)!=0)×1, where Ngx is the GPU grid size in the X direction (see FIG. 2) and NOP the number of pixels. The signs % and !=stand for remaining and opposed to, respectively. The GPU grid size in the Y direction is equal to the number of receiver elements Nr. This indicates that each row of grids calculates the correct travel-times for each receiving element in the ultrasound probe 2.

The grid used for the reconstruction kernel is three-dimensional. The first two dimensions are the same as the ray-tracing kernel, and the third one (in the Z direction) is the number of transmitter elements Nt in the ultrasound probe 2.

Thus, the ray tracing kernel 21 has a two dimensional grid, with a first dimension equal to N_{gx=NOPblock.x}+((NOP % block.x)!=0)×1, and a second dimension equal to the number Nr of receiver elements; and the reconstruction kernel 22 has a three dimensional grid, comprising the two-dimensional grid of the ray tracing kernel 21 plus a third dimension equal to the number Nt of transmitter elements of the ultrasound probe 2. Note that other GPU architectures could be used as well considering the flexibility of CUDA in defining grid sizes in different directions.

To calculate the round-trip travel-times that are required to reconstruct the image with a delay-and-sum algorithm, both the transmit and receive travel-times are used. In a full synthetic aperture imaging, SAI, scheme, the number of transmitter elements Nt and receiver elements Nr and their coordinates are the same. Therefore, the receive travel-times calculated with respect to Nr receivers (see the ray tracing kernel 21 in FIG. 2) can be used as transmit travel-times as well. Thus, in a further embodiment, sending and detecting the ultrasound signal is applied in accordance with a synthetic aperture imaging, SAI, scheme.

In practice, SAI is likely not the most optimal imaging strategy for TUI due to 1) a low signal-to-noise ratio (i.e., transmitting with a single element generates a low acoustic pressure in the brain) and 2) a low data acquisition frame rate, which does not allow e.g. blood flow quantification in the brain (usually used for diagnosis of brain disorders). To address these issues, in further embodiments, sending and detecting the ultrasound signal is applied in accordance with a multi angle plane wave imaging, MA-PWI, scheme, or a multi angle diverging wave imaging, MA-DWI, scheme. MA-PWI and MA-DWI schemes have a great potential since a good image quality can be achieved while a faster data acquisition allows capturing transient phenomena in the brain, and transmitting with sub-apertures or with all the elements of the probe (with appropriate transmit delays) ensures a good signal-to-noise ratio (SNR). Using MA-PWI/DWI schemes requires estimating the transmit travel-times from virtual transmitters (usually defined behind the ultrasound probe) to pixels. This requires an additional ray tracing in steps 2, 4 and 6 as described above. To implement this, the ray tracing kernel 21 in FIG. 2 may be configured with a Z grid size equal to the number of virtual transmitters. MA-PWI offers faster image reconstruction compared to SAI. That is because the size of the third dimension (Z) of the reconstruction kernel 22 (see FIG. 2) is equal to the number of virtual transmitter element (e.g. from 3 to 15), and less load/copy transactions from the global memory 20 to on-chip memories 18, and the other way around, are needed. Also the additional ray tracing (for estimating the transmit travel-times) is not computationally expensive.

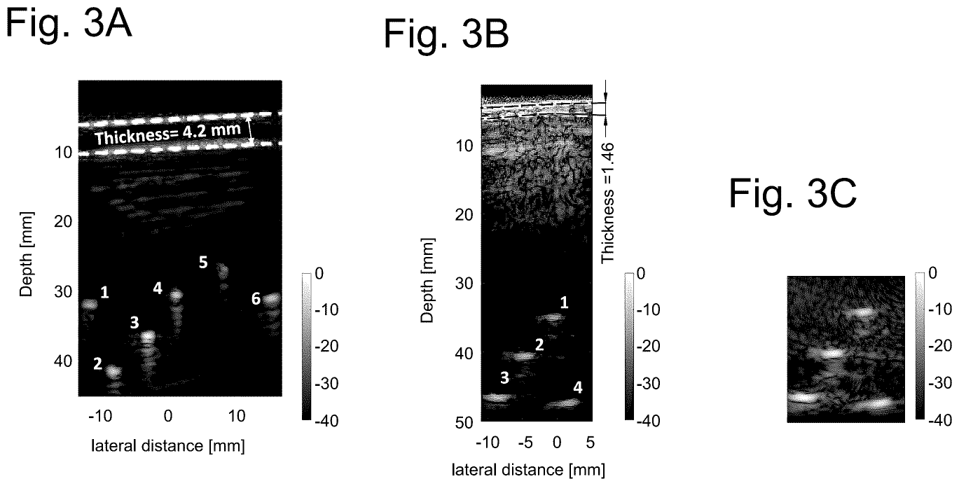

FIG. 3A and FIGS. 3B and C show an example of an image acquired using one of the present invention embodiments for acquiring an image through a sample of Sawbones plate and human temporal bone, respectively, with 50 μm wires held below, acting to mimic scatterers in a human brain (target area 6). The dashed lines show the near and far surfaces of the bone (first element 5), and the numbered spots in the image are the detected wires. It was shown that the accuracy of the near and far surfaces, and the position of the (wire) scatterers in the image as obtained was very high. Moreover, processing times observed were sufficiently low to enable real-time observations. The processing time (frame rate) with a pixel size of 100 μm is higher (lower) than 200 μm. Using SAI (MA-PWI) schemes to reconstruct images up to depths of 20 mm and 70 mm, a frame rate of 32 Hz (40 Hz) and 19 Hz (32 Hz) were obtained for a pixel size of 200 μm.

The above described exemplary embodiments can also be summarized in the following numbered and interrelated embodiments:
Embodiment 1. Method for transcranial ultrasound imaging (TUI), comprising:
sending an ultrasound wave to a target imaging area (6) through a superficial area (4) and a first element (5), where the target imaging area (6), the superficial area (4) and the first element (5) have different ultrasound wave speeds;
detecting ultrasound waves reflected or backscattered from the target imaging area (6) through the first element (5) and the superficial area (4), and detecting ultrasound waves reflected or backscattered from the first element (5) through the superficial area (4); and processing the detected ultrasound waves by:
   using estimates of the wave speed in the superficial area (4), in the first element (5) and in the target imaging area (6); and
   using the detected ultrasound waves reflected or backscattered from the first element (5) through the superficial area (4) for forming the ultrasound image of the superficial area (4) up to the near surface of the first element (5); and
   determining the position and geometry of the near surface of the first element (5); and
   applying a phase aberration correction to the detected ultrasound waves reflected or backscattered from the first element (5) by defining intermediate points on the near surface of the first element (5) to test different ultrasound ray paths, for forming the ultrasound image of the first element (5) up to the far surface of the first element (5), using the estimates of wave speed, and the position and geometry of the near surface of the first element (5); and
   determining the position and geometry of the far surface of the first element (5); and
   applying a phase aberration correction to the detected ultrasound waves reflected or backscattered from the target imaging area (6) by defining intermediate points on the far surface of the first element (5) to test different ultrasound ray paths, for forming the ultrasound image of the target imaging area (6) using the estimates of wave speed, and the position and geometry of the near and far surfaces of the first element (5).
wherein the phase aberration correction includes the evaluation of the travel times for multiple ultrasound ray paths passing through the intermediate points defined on the near and far surfaces of the first element (5), and determines the correct ultrasound ray path by selecting the ultrasound ray path with the shortest travel time.
Embodiment 2. Method according to embodiment 1, wherein the ultrasound waves are sent and detected using an ultrasound probe (2) having a first plurality Nt of transmitter elements and a second plurality Nr of receiver elements, and the phase aberration correction comprises a two-point ray tracing technique determining a shortest travel time connecting a pixel in the element (4), or inside the first element (5), or in the target imaging area (6), to a source or receiver element of the ultrasound probe (2), for each of the first plurality Nt of transmitter elements and for each of the second plurality Nr of receiver elements, via one intermediate point per crossed interface.
Embodiment 3. Method according to embodiment 2, wherein the phase aberration correction comprises applying a ray-tracing kernel (21) and a reconstruction kernel (22) consecutively, first for finding intermediate points on the near surface of the first element (5), then for finding intermediate points on the far surface of the first element (5), and, finally, to generate the transcranial ultrasound image.
Embodiment 4. Method according to any one of embodiments 1-3, further including applying a phase aberration correction to the detected ultrasound signal for a second element (4), the second element (4) having predetermined acoustic properties.
Embodiment 5. Method according to any one of embodiments 2-4, wherein the ultrasound probe (2) further includes an acoustic lens (3).
Embodiment 6. Method according to any one of embodiments 2-5, wherein the ultrasound probe (2) comprises a one-dimensional array or a two-dimensional array.
Embodiment 7. Method according to any one of embodiments 1-6, wherein the steps of processing are executed on a graphical processing unit, GPU (13), a cluster processing system, and/or a central processing unit (CPU).
Embodiment 8. Method according to any one of embodiments 1-7, wherein sending and detecting the ultrasound signal is applied in accordance with a synthetic aperture imaging, SAI, scheme.
Embodiment 9. Method according to any one of embodiments 1-7, wherein sending and detecting the ultrasound signal is applied in accordance with a multi angle plane wave imaging, MA-PWI, scheme, or a multi angle diverging wave imaging, MA-DWI, scheme.
Embodiment 10. System for transcranial ultrasound imaging (TUI), comprising
an ultrasound unit (11) having an ultrasound probe (2) with a plurality of transmitter and receiver elements for sending and detecting an ultrasound signal;
a processing unit (12) connected to the ultrasound unit (11), and arranged for data manipulation and image display; and
a graphic processing unit (13) connected to the processing unit (12) and arranged to execute the method according to any one of embodiments 1-9.

The present invention has been described above with reference to a number of exemplary embodiments as shown in the drawings. Modifications and alternative implementations of some parts or elements are possible, and are included in the scope of protection as defined in the appended embodiments.

1. A method for transcranial ultrasound imaging, comprising:
sending, by an ultrasound probe, an ultrasound wave to a target imaging area through a superficial area and a first element, wherein the ultrasound wave travels through the target imaging area, the superficial area and the first element at different ultrasound wave speeds;
detecting, by the ultrasound probe, ultrasound waves reflected or backscattered from the target imaging area through the first element and the superficial area, and detecting, by the ultrasound probe, ultrasound waves reflected or backscattered from the first element through the superficial area;
processing the detected ultrasound waves by:
using estimates of the wave speed in the superficial area, in the first element and in the target imaging area; and
using the detected ultrasound waves reflected or backscattered from the first element through the superficial area for forming the ultrasound image of the superficial area until the near surface of the first element; and
determining the position and geometry of the near surface of the first element; and
applying a phase aberration correction to the detected ultrasound waves reflected or backscattered from the first element by defining intermediate points on the near surface of the first element to test different ultrasound ray paths, for forming the ultrasound image of the first element until the far surface of the first element, using the estimates of wave speed, and the position and geometry of the near surface of the first element; and
determining the position and geometry of the far surface of the first element; and
applying a phase aberration correction to the detected ultrasound waves reflected or backscattered from the target imaging area by defining intermediate points on the far surface of the first element to test different ultrasound ray paths, for forming the ultrasound image of the target imaging area using the estimates of wave speed, and the position and geometry of the near and far surfaces of the first element;
wherein the phase aberration correction includes the evaluation of the travel times for multiple ultrasound ray paths passing through the intermediate points defined on the near and far surfaces of the first element, and determines the correct ultrasound ray path by selecting the ultrasound ray path with the shortest travel time.

2. The method according to claim 1, wherein the ultrasound waves are sent and detected using an ultrasound probe having a first plurality Nt of transmitter elements and a second plurality Nr of receiver elements, and the resource-efficient ray-tracing technique comprises a two-point ray tracing technique determining a shortest travel time connecting a pixel in the element, or inside the first element, or in the target imaging area, to a source or receiver element of the ultrasound probe, for each of the plurality Nt of sources and for each of the plurality Nr of receiver elements, via one intermediate point per crossed interface.

3. The method according to claim 2, wherein the ray-tracing technique comprises applying a ray-tracing kernel and a reconstruction kernel consecutively, first for finding intermediate points on a near surface of the first element, then for finding intermediate points on the far surface of the first element, and, finally, to generate the transcranial ultrasound image.

4. The method according to claim 1, further including applying a phase aberration correction to the detected ultrasound signal for a second element, the second element having predetermined acoustic properties.

5. The method according to claim 2, wherein the ultrasound probe further includes an acoustic lens.

6. The method according to claim 2, wherein the ultrasound probe comprises a one-dimensional array or a two-dimensional array.

7. The method according to claim 1, wherein the steps of processing are executed on a graphical processing unit (GPU), a cluster processing system, and/or a central processing unit (CPU).

8. The method according to claim 1, wherein sending and detecting the ultrasound signal is applied in accordance with a synthetic aperture imaging (SAI) scheme.

9. The method according to claim 1, wherein sending and detecting the ultrasound signal is applied in accordance with a multi angle plane wave imaging (MA-PWI) scheme, or a multi angle diverging wave imaging (MA-DWI) scheme.

10. System A system for transcranial ultrasound imaging (TUI), comprising
an ultrasound unit having an ultrasound probe with a plurality of transmitter and receiver elements for sending and detecting an ultrasound signal;
a processing unit connected to the ultrasound unit, and arranged for data manipulation and image display; and
a graphic processing unit connected to the processing unit and arranged to execute the method according to claim 1.
